(19) United States
(12) Patent Application Publication
(10) Pub. No.: US 2020/0335214 A1
(43) Pub. Date: Oct. 22, 2020

(54) METHOD FOR PREDICTING OBESITY RELATED DISEASE USING IMAGES OF THE SUBCUTANEOUS ADIPOSE TISSUE OR THE EPIDIDYMAL ADIPOSE TISSUE

(71) Applicants: INSTITUT NATIONAL DE LA SANTE ET DE LA RECHERCHE MEDICALE (INSERM), Paris (FR); UNIVERSITÉ PARIS DIDEROT - PARIS 7, Paris (FR); ASSISTANCE PUBLIQUE - HOPITAUX DE PARIS, Paris (FR)

(72) Inventors: Philippe GARTEISER, Asnieres (FR); Benjamin LEPORQ, Lyon (FR); Bernard VAN BEERS, Boulogne (FR)

(21) Appl. No.: 16/303,696

(22) PCT Filed: May 24, 2017
(86) PCT No.: PCT/EP2017/062675

§ 371 Date: Nov. 21, 2018

(30) Foreign Application Priority Data
May 25, 2016 (EP) 16305608.8

(51) Int. Cl. G16H 50/20; G16H 50/30; G16H 50/50; A61B 5/055; A61B 5/00; G01R 33/48; G01R 33/563; G06T 11/00

(52) U.S. Cl. CPC G16H 50/20; G16H 50/30; G16H 50/50; A61B 5/055; G06T 2207/30004; G01R 33/4828; G01R 33/56358; G06T 11/008; G06T 2207/10096; A61B 5/4872

(57) ABSTRACT

The present invention relates to obesity related diseases, such as cancer of non-alcoholic fatty liver disease (NAFLD). Tissue perfusion is currently investigated by using dynamic contrast-enhanced magnetic resonance imaging which is an invasive technique and does not provide enough accuracy. As a result, the inventors worked on post-processing images of subcutaneous adipose tissue or the epididymal adipose tissue taken with a magnetic resonance imaging technique and a multifrequency magnetic resonance elastography technique to obtain parameters such as loss modulus and storage modulus which are more accurate for a diagnosing purpose. This post-processing method may be applied for a method for predicting that a subject is at risk of suffering from said disease, identifying a therapeutic target or a biomarker and screening compounds useful as medicine.

### TECHNICAL FIELD OF THE INVENTION

The invention concerns a method for predicting that a subject is at risk of suffering from an obesity related disease. The invention also relates to a method for diagnosing an obesity related disease. The invention also concerns a method for identifying a therapeutic target for preventing and/or treating an obesity related disease. The invention also relates to a method for identifying a biomarker, the biomarker being a diagnostic biomarker of an obesity related disease, a susceptibility biomarker of an obesity related disease, a prognostic biomarker of an obesity related disease or a predictive biomarker in response to the treatment of an obesity related disease. The invention also concerns a method for screening a compound useful as a medicine, the compound having an effect on a known therapeutical target, for preventing and/or treating an obesity related disease. The invention also relates to the associated computer program products and a computer readable medium.

### BACKGROUND OF THE INVENTION

An obesity related disease is a cancer, type 2 diabetes, a heart disease, a liver disease or a non-alcoholic fatty liver diseases (NAFLD). Such kinds of diseases concern a high number of people in the world.

There is therefore a need to be able to predict with accuracy the risk for a subject to suffer from this disease.

The main issue comes from a double choice: the choice of the properties to be measured and the choice of the techniques to be used for measuring the properties.

For instance, for liver disease, it is desirable to obtain information on the tissue cellularity, their perfusion, the degree of molecular transport in hepatocytes and their visco-elasticity.

As an example, the tissue perfusion may be investigated by using dynamic contrast-enhanced MRI, or perfusion computed tomography and the transport function of the hepatocytes may be measured by using gadoxetate MRI enhancement analyses or blood tests such as aminotransferase, bilirubin or gamma glutamyl transferase levels.

However, some of these techniques are invasive and do not provide in combination a good accuracy in the prediction of the risk for a subject to suffer from an obesity related disease.

### SUMMARY OF THE INVENTION

The invention aims at providing a method which involves less invasive technique and provides the best accuracy in the prediction of the risk for a subject to suffer from an obesity related disease.

To this end, the specification describes a method for predicting that a subject is at risk of suffering from an obesity related disease, the method for predicting at least comprising the step of carrying out the steps of a method for post-processing images of the subject, to obtain determined parameters and the step of predicting that the subject is at risk of suffering from the obesity related disease based on the determined parameters.

The specification also relates to a method for diagnosing an obesity related disease, the method for diagnosing at least comprising the step of carrying out the steps of a method for post-processing images of the subject, to obtain determined parameters and the step of diagnosing the obesity related disease based on the determined parameters.

The specification also concerns a method for identifying a therapeutic target for preventing and/or treating an obesity related disease, the method comprising the step of carrying out the steps of a method for post-processing images of a first subject, to obtain first determined parameters corresponding to the determined parameters for the first subject, the first subject being a subject suffering from the obesity related disease. The method for identifying further comprising the step of carrying out the steps of the method for post-processing images of a second subject, to obtain second determined parameters corresponding to the determined parameters for the second subject, the second subject being a subject not suffering from the obesity related disease, and the step of selecting a therapeutic target based on the comparison of the first and second determined parameters.

The specification also relates to a method for identifying a biomarker, the biomarker being a diagnostic biomarker of an obesity related disease, a susceptibility biomarker of an obesity related disease, a prognostic biomarker of an obesity related disease or a predictive biomarker in response to the treatment of an obesity related disease, the method comprising the step of carrying out the steps of a method for post-processing images of a first subject, to obtain first determined parameters, the first determined parameters corresponding to the determined parameters for the first subject, the first subject being a subject suffering from the obesity related disease. The method for identifying further comprising the step of carrying out the steps of the method for post-processing images of a second subject, to obtain second determined parameters corresponding to the determined parameters for the second subject, the second subject being a subject not suffering from the obesity related disease, and the step of selecting a biomarker based on the comparison of the first and second determined parameters.

The specification also concerns a method for screening a compound useful as a medicine, the compound having an effect on a known therapeutical target, for preventing and/or treating an obesity related disease, the method comprising a step of carrying out the steps of a method for post-processing images of a first subject, to obtain first determined parameters, the first determined parameters corresponding to the determined parameters for the first subject, the first subject being a subject suffering from the obesity related disease and having received the compound. The method also comprises a step of carrying out the steps of the method for post-processing images of a second subject, to obtain second determined parameters, the second determined parameters corresponding to the determined parameters for the second subject, the second subject being a subject suffering from the obesity related disease and not having received the compound. The method also comprises a step of selecting a compound based on the comparison of the first and second determined parameters.

Each of the method for predicting, method for diagnosing, method for identifying a therapeutic target, method for identifying a biomarker and the method for screening a compound useful as a medicine involves a method for post-processing. The method for post-processing is a method for post-processing two series of images of tissues of a subject, the tissues being either the subcutaneous adipose tissue or the epididymal adipose tissue, the first series of images being taken by a magnetic resonance imaging technique, the magnetic resonance imaging technique involving successive echoes of a multiple-gradient echo sequence with bipolar readout gradients and the second series of images being taken by using a multifrequency magnetic resonance elastography technique. The method for post-processing comprising the substeps of quantifying the proportion of unsaturated fatty acids and the proportion of saturated fatty acids in the tissues of the subject based on a first analysis applied on the first series of images and calculating the values of the storage modulus and the loss modulus in the tissues of the subject based on a second analysis applied on the second series of images. The determined parameters are the quantified proportion of unsaturated fatty acids, the quantified proportion of saturated fatty acids, the calculated values of the storage modulus and the calculated loss modulus.

Such method for post-processing should be construed as a computer-implemented method for post-processing two series of images of tissues of a subject, the tissues being either the subcutaneous adipose tissue or the epididymal adipose tissue, the first series of images being images resulting from a magnetic resonance imaging technique, the magnetic resonance imaging technique involving successive echoes of a multiple-gradient echo sequence with bipolar readout gradients and the second series of images being images resulting from a multifrequency magnetic resonance elastography technique.

In other words, the method for post-processing is to be interpreted as a method wherein images are provided and processed.

Thanks to the invention, two non-invasive techniques combined with specific analysis enable to characterize an obesity related disease more efficiently.

According to further aspects of the invention which are advantageous but not compulsory, any method chosen among the previously described method for predicting, method for diagnosing, method for identifying a therapeutic target, method for identifying a biomarker and the method for screening a compound useful as a medicine might incorporate one or several of the following features, taken in any technically admissible combination:
each image of the first series of images associates to each pixel of the image the amplitude of the measured signal in the magnetic resonance imaging technique and the phase of the measured signal in the magnetic resonance imaging technique, the first analysis applied on the first series of images at the substep of quantifying comprising unwrapping the phase of each image, to obtain unwrapped images, extracting a complex signal over echo time for at least one pixel of the unwrapped images, to obtain at least one extracted complex signal, and calculating fat characterization parameters by using a fitting technique applied on a model, the model being a function which associates to a plurality of parameters each extracted complex signal, the plurality of parameters comprising at least two fat characterization parameters, the magnitude error generated by the use of the bipolar readout gradients and the phase error generated by the use of the bipolar readout gradients, the fitting technique being a non-linear least-square fitting technique using pseudo-random initial conditions, and quantifying the proportion of unsaturated fatty acids and proportion of saturated fatty acids in the area of the subject based on the calculated fat characterization parameters.
the fat characterization parameters are chosen in the group consisting of the number of double bounds, the number of methylene-interrupted double bounds and the chain length.
the first analysis applied on the first series of images at the substep of quantifying further comprises the step of determining the phase jump in the phase between two images, the first image being taken at a first echo and the second image being taken at a second consecutive echo, comparing the phase jump with a threshold value, and correcting the phase value when the phase jump is superior or equal to the threshold value.
the model further depends on the complex intensity of water, the complex intensity of fat and a complex field map taking into account the effect of transversal relaxivity rate and the field inhomogeneity in the magnetic field used in the magnetic resonance imaging technique.
the first analysis applied on the first series of images at the substep of quantifying further comprises determining the proton density fat fraction and the fatty acid composition based on the calculated fat characterization parameters.
each image of the second series of images associates to each pixel of the image the amplitude of the measured signal in the magnetic resonance elastography technique and the phase of the measured signal in the magnetic resonance elastography technique, the second analysis at the substep comprises unwrapping the phase of each image, to obtain unwrapped images, obtaining the propagation equation of a wave in the imaged tissues based on the unwrapped images, and recovering the values of the storage modulus and the loss modulus in the tissues of the subject by using the propagation equation and the amplitude of the measured signal.
the substep of calculating the values of the storage modulus and the loss modulus in the tissues of the subject based on a second analysis applied on the second series of images further comprises determining the dispersion coefficients of the calculated values of the storage modulus, the calculated loss modulus and the determined parameters further comprises the dispersion coefficients of the calculated values of the storage modulus, the calculated loss modulus.
the dispersion coefficients are respectively the variation of the values of the storage modulus with frequency and the variation of the values of the loss modulus with frequency.

The specification also describes a computer program product comprising a computer readable medium, having thereon a computer program comprising program instructions, the computer program being loadable into a data-processing unit and adapted to cause execution of a method chosen among the previously described method for predicting, method for diagnosing, method for identifying a therapeutic target, method for identifying a biomarker and the method for screening a compound useful as a medicine when the computer program is run by the data processing unit

The specification also describes a computer readable medium having encoded thereon a computer program as previously described.

### BRIEF DESCRIPTION OF THE DRAWINGS

The invention will be better understood on the basis of the following description which is given in correspondence with the annexed figures and as an illustrative example, without restricting the object of the invention. In the annexed figures:

FIG. 1 shows schematically a system and a computer program product whose interaction enables to carry out a method for predicting that a subject is at risk of suffering from an obesity related disease, and

FIGS. 2 to 39 are relative to an experiment carried out by the Applicant.

### DETAILED DESCRIPTION OF SOME EMBODIMENTS

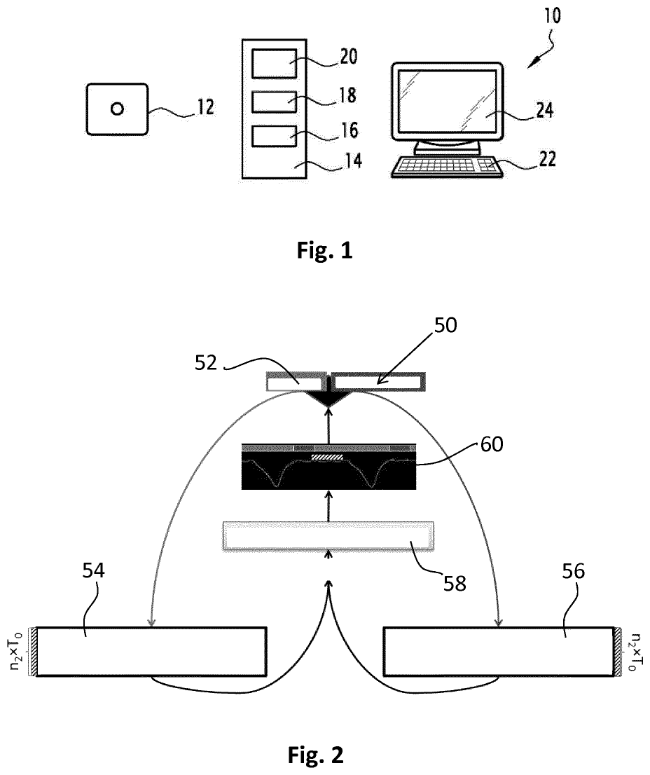

A system 10 and a computer program product 12 are represented in FIG. 1. The interaction between the computer program product 12 and the system 10 enables to carry out a method for predicting that a subject is at risk of suffering from an obesity related disease.

System 10 is a computer. In the present case, system 10 is a laptop.

More generally, system 10 is a computer or computing system, or similar electronic computing device adapted to manipulate and/or transform data represented as physical, such as electronic, quantities within the computing system's registers and/or memories into other data similarly represented as physical quantities within the computing system's memories, registers or other such information storage, transmission or display devices.

System 10 comprises a processor 14, a keyboard 22 and a display unit 24.

The processor 14 comprises a data-processing unit 16, memories 18 and a reader 20. The reader 20 is adapted to read a computer readable medium.

The computer program product 12 comprises a computer readable medium.

The computer readable medium is a medium that can be read by the reader of the processor. The computer readable medium is a medium suitable for storing electronic instructions, and capable of being coupled to a computer system bus.

Such computer readable storage medium is, for instance, a disk, a floppy disks, optical disks, CD-ROMs, magnetic-optical disks, read-only memories (ROMs), random access memories (RAMs) electrically programmable read-only memories (EPROMs), electrically erasable and programmable read only memories (EEPROMs), magnetic or optical cards, or any other type of media suitable for storing electronic instructions, and capable of being coupled to a computer system bus.

A computer program is stored in the computer readable storage medium. The computer program comprises one or more stored sequence of program instructions.

The computer program is loadable into the data-processing unit and adapted to cause execution of the method for post-processing images when the computer program is run by the data-processing unit.

Operation of the system 10 is now described by illustrating an example of carrying out the method for predicting that a subject is at risk of suffering from an obesity related disease.

The subject is, for instance, a mammal.

According to specific embodiments, the subject is a small animal, such as a rabbit or a mouse.

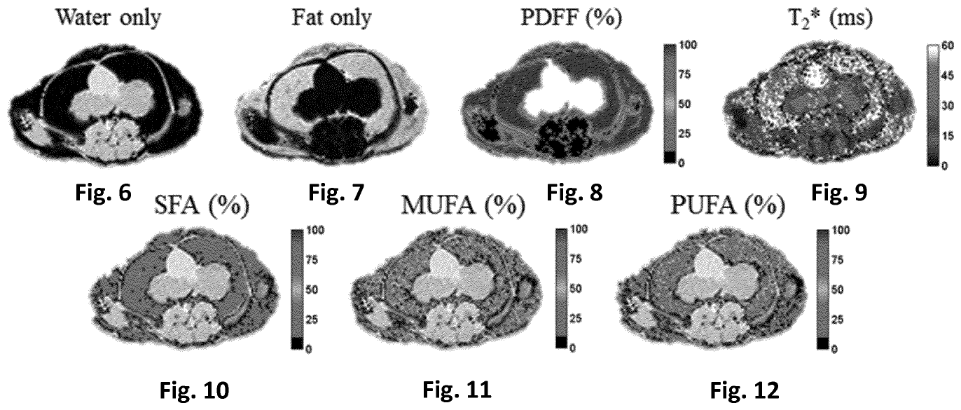

In the experience described in reference with FIGS. 2 to 39, the subjects are mice.

An obesity related disease is a cancer, type 2 diabetes, a heart disease, a liver disease or a non-alcoholic fatty liver disease (NAFLD). Nonalcoholic fatty liver disease (NAFLD) and its most severe form, nonalcoholic steatohepatitis (NASH), are associated with high fat diet, high triglyceride levels, obesity, the metabolic syndrome and type II diabetes, and pose an increased risk of cardio vascular disease. NAFLD is an accumulation of fat in the liver that is not a result of excessive consumption of alcohol. 15% to 25% of cases of NAFLD progress and are associated with inflammation and liver damage; this condition is referred to as NASH. NASH is associated with an increased risk of developing liver cirrhosis and subsequence complications, including hepatocellular carcinoma.

The method for predicting that a subject is at risk of suffering from an obesity related disease comprises two steps: a step S10 of carrying out the steps of a method for post-processing images of the subject, to obtain determined parameters and a step S20 of predicting that the subject is at risk of suffering from the obesity related disease based on the determined parameters.

At the step S10, a method of post-processing is carried out on two series of images of tissues of the subject.

The tissues considered at the step S10 are specific tissues, which are either the subcutaneous adipose tissue or the epididymal adipose tissue.

The first series of images are taken or acquired with a magnetic resonance imaging technique.

The magnetic resonance imaging technique involves successive echoes of a multiple-gradient echo sequence with bipolar readout gradients.

According to the specific embodiment described, the multiple-gradient echo sequence is a spoiled gradient echo sequence.

In addition, the magnetic resonance imaging technique is carried out by a preclinical system operating at magnetic field with a magnitude of 7.0 Tesla (T).

Each image associates to each pixel of the image the amplitude of the measured signal in the magnetic resonance imaging technique and the phase of the measured signal in the magnetic resonance imaging technique.

In other words, for each image, it can be defined a magnitude map and a phase map.

The second series of images are acquired by using a multifrequency magnetic resonance elastography technique.

The method for processing comprises the substep of quantifying the proportion of unsaturated fatty acids and the proportion of saturated fatty acids in the tissues of the subject based on a first analysis applied on the first series of images.

The first analysis comprises four operations, which are an operation of correcting, an operation of extracting, an operation of calculating and an operation of quantifying.

At the operation of correcting, a mask is built from the magnitude images to suppress background and air cavities to decrease overall computing time by reducing the number of processed pixels.

Segmentation is performed with an active contour approach which is based on techniques of curve evolution, Mumford-Shah function for segmentation and level sets. This method includes a model able to detect objects which boundaries are not necessarily defined by gradients.

At the operation of correcting, the phase of each image is unwrapped to obtain unwrapped images.

For this, the phase jump is determined in the phase between two images, the first image being acquired at a first echo and the second image being acquired at a second consecutive echo. The phase jump is compared with a threshold value, and the phase value is corrected when the phase jump is superior or equal to the threshold value.

More precisely, a phase-time array was extracted pixel by pixel and individual points were corrected for wrap by adding multiples of ±2π when absolute jumps between consecutive elements of the array were greater than or equal to a jump tolerance of π radians.

Then, corrected complex images are generated from the magnitude and the phase unwrapped images.

At the end of the correcting operation, corrected complex images are obtained.

At the operation of extracting, a complex signal over echo time for at least one pixel of the unwrapped images is extracted.

According to the specific embodiment described, from multiple echo unwrapped complex images, a complex signal over echo time S(tₙ, x, y) is extracted pixel by pixel, tᵣ, being the time of the echo and x, y being spatial coordinates.

The complex signal over echo time corresponds to the complex gradient echo signal at time tₙ.

At the end of the extracting operation, for each pixel, the complex signal over echo time S(tₙ, x, y) is known.

Optionally, at the correcting operation, phase images for zero (time independent) and first order (time-dependent) dephasings are also corrected.

Alternatively, the first analysis comprises an operation of providing corrected real images to be post-processed. At the operation of calculating, fat characterization parameters are calculated by using a fitting technique applied on a model.

The model is a model for the complex gradient echo signal at time tₙ, from a pixel containing water and fat with an unknown number of spectral components.

In other words, such model is a function which associates to a plurality of parameters each extracted complex signal.

The plurality of parameters comprises at least two fat characterization parameters, the magnitude error generated by the use of the bipolar readout gradients and the phase error generated by the use of the bipolar readout gradients.

Fat characterization parameters may be any parameters which enable(s) to obtain information on the chemical structure of fat.

According to a specific embodiment, the fat characterization parameters are chosen in the group consisting of the number of double bounds ndb, the number of methylene-interrupted double bounds nmidb and the chain length CL.

The model is based on eight separate fat resonances.

For instance, the model is the following model:

$$\check{S} ( {t}_{n , x , y} ) = ( \check{W} {n}_{w} + \check{F} {\sum }_{k = 1}^{8} {n}_{k} ( ndb , CL , nmidb ) {e}^{2 π {if}_{{kt}_{n}}} ) {e}^{2 π i \check{ψ} {t}_{n}} {e}^{{( - 1 )}^{n} i \check{θ} ( x , y )}$$

where:
Ŵ is the complex intensity of water,
n_{w} is the number of protons in the water peak
\hat{F} is the complex intensity of fat,
nₖ(ndb, CL, nmidb) is the number of protons in the fat spectrum component k according to the number of double bounds ndb, the number of methylene-interrupted double bounds nmidb and the chain length CL,
Fₖ is the frequency shift of the kth fat resonance with relation to water (considered on-resonance),
\hat{ψ} is a complex field map taking into account the effect of transversal relaxivity rate and the field inhomogeneity in the magnetic field used in the magnetic resonance imaging technique,
\hat{θ}(x, y) being a complex error map depending from the magnitude error generated by the use of the bipolar readout gradients and the phase error generated by the use of the bipolar readout gradients

It can be noticed that the model further depends on the complex intensity of water Ŵ, the complex intensity of fat \hat{F} and a complex field map \hat{ψ} taking into account the effect of transversal relaxivity rate and the field inhomogeneity in the magnetic field used in the magnetic resonance imaging technique.

\hat{ψ} is a complex field map summarizing the effect of both R2* (1/T2*) and B₀ field inhomogeneity according to:

\hat{ψ}=Δ+iR*₂

where R2* is the transversal relaxivity rate and Δ the B₀ field inhomogeneity.

To take into account the phase error and the amplitude modulation caused by the bipolar readout gradients, a complex error map \hat{θ}(x, y) was included in the model. This latter induces a modulation in the signal according to the echo n polarity. This complex error map summarized both phase ϕ and magnitude error ε according to the following linear relationship:

θ=Φ−iε

Therefore, in the example, the model depends from eleven parameters, which are the complex intensity of water Ŵ, the complex intensity of fat \hat{F}, the number of double bounds ndb, the number of methylene-interrupted double bounds nmidb, the chain length CL, two parameters via the complex field map \hat{ψ}, the magnitude error generated by the use of the bipolar readout gradients and the phase error generated by the use of the bipolar readout gradients via the complex error map \hat{θ}.

To obtain these eleven parameters, a fitting technique was used.

The fitting technique a non-linear least-square fitting technique using pseudo-random initial conditions.

As an example, the eleven parameters may be derived by using a non-linear least-square fit using the multi-start Levenberg-Marquardt algorithm.

In mathematics and computing, the Levenberg-Marquardt algorithm (LMA), also known as the damped least-squares method, is used to solve non-linear least squares problems. These minimization problems arise especially in least squares curve fitting.

The LMA interpolates between the Gauss-Newton algorithm (GNA) and the method of gradient descent. The LMA is more robust than the GNA, which means that in many cases it finds a solution even if it starts very far off the final minimum. For well-behaved functions and reasonable starting parameters, the LMA tends to be a bit slower than the GNA. LMA can also be viewed as Gauss-Newton using a trust region approach.

The LMA is a very popular curve-fitting algorithm used in many software applications for solving generic curve-fitting problems. However, as for many fitting algorithms, the LMA finds only a local minimum, which is not necessarily the global minimum.

A multi-start technique or the use of pseudo-random initial conditions corresponds to the use of a grid of pseudo-random initial conditions. This enables to improve the robustness of optimization and avoid multiple local minima problem.

In other words, the fitting technique is carried out a certain number of times, each time corresponding to different initial conditions.

For instance, the number of times is superior or equal to five, preferably superior or equal to ten, more preferably superior or equal to twenty.

According to a specific example, the number of times is equal to twenty.

Optionally, to decrease the degree of freedom, the chain length CL is expressed according to the number of double bounds ndb using a heuristic approximation. Such heuristic approximation is usually a linear relationship.

At the end of the calculating operation, the fat characterization parameters are obtained.

At the operation of quantifying, the proportion of unsaturated fatty acids and proportion of saturated fatty acids in the region of interest in the subject are obtained based on the calculated fat characterization parameters.

Preferably, at the operation of quantifying, the proportions of saturated, monounsaturated and polyunsaturated fatty acids in the region of interest in the subject are quantified based on the calculated fat characterization parameters.

As an example, the quantifying operation comprises determining the proton density fat fraction and the fatty acid composition based on the calculated fat characterization parameters.

According to a specific embodiment, the proton density fat fraction (PDFF) is calculated by using the following formula:

$$PDFF = \frac{‖ \check{F} ‖}{‖ \check{W} ‖ + ‖ \check{F} ‖}$$

For determining the fatty acid composition, it is proposed to use the following relations:

$$UFA = \frac{n d b - n m i d b}{3}$$

$$and$$

$$PUFA = \frac{n m i d b}{3}$$

Where:
UFA is the unsaturated fatty acid fraction in %, and
PUFA is the polyunsaturated fatty acid fraction in %.

Optionally, determining the fatty acid composition may also comprise deducing the monounsaturated fatty acid fraction, which is generally labeled MUFA. For this, the following equation may be used:

MUFA=UFA−PUFA

Optionally, determining the fatty acid composition may also comprise calculating the saturated fatty acid fraction, which is generally labeled SFA. For this, the following equation may be used:

SFA=100−UFA

The method for processing images comprises the substep of calculating the values of the storage modulus and the loss modulus in the tissues of the subject based on a second analysis applied on the second series of images.

Storage and loss modulus are defined as the real and imaginary parts of the complex shear modulus. Storage modulus corresponds to the elastic term μ (in kilopascals) of the shear component of the wave propagation equation. This wave propagation equation can be written as:

ρ∂ₜ²u=μΔu+(λ+μ)∇(∇u)+η∂ₜΔu+(ξ+η)∂ₜ∇(∇u)

Loss modulus corresponds to the viscous term of the shear component of the wave propagation equation, η, multiplied by the frequency of the mechanical wave used for the mechanical actuation of the tissue.

The tissues are the subcutaneous adipose tissue and the epididymal adipose tissue.

Anatomically, the two adipose tissue compartments can be delimited using anatomical landmarks, such as the position relative to the peritoneum. Subcutaneous fat is located on the outside of the peritoneum. Epididymal fat is located on the inside of the peritoneum, in the lower abdominal cavity.

It is recalled that the each image of the second series of images is taken with a multifrequency magnetic resonance elastography technique.

The magnetic resonance elastography technique was applied using a spin echo sequence. Application of motion encoding gradients enable to encode, in the phase of the MRI signal, the instantaneous position of tissue units (voxels) as they oscillate around a central position during the continuous application of a mechanical stimulation in the form of an acoustic wave with a narrow frequency content. Acquisition of the instantaneous position at various time offsets of the mechanical oscillation can be achieved for instance by repeating the acquisition with increasing values of a delay between start of acquisition and mechanical actuation.

The magnetic resonance elastography technique is qualified as “multifrequency” because several mechanical frequencies are probed.

For instance, two mechanical frequencies are probed, the ratio between the mechanical frequencies being inferior to 2.

The magnetic resonance elastography technique also comprises a respiratory motion compensation.

An example is described below in reference to FIG. 2. Each image associates to each pixel of the image the amplitude of the measured signal in the magnetic resonance elastography technique and the phase of the measured signal in the magnetic resonance elastography technique.

The second analysis comprises unwrapping the phase and calculating the storage modulus, G′ and the loss modulus, G″.

The calculating is achieved by solving the plane wave propagation equation under assumption of local homogeneity and by filtering the obtained solutions.

According to a specific embodiment, the filtering comprises removing the compressional components and applying a Gaussian filter. Compressional components correspond to the changes in tissue volume caused by the mechanical wave.

The determined parameters obtained at the step S10 are therefore the quantified proportion of unsaturated fatty acids, the quantified proportion of saturated fatty acids, the calculated values of the storage modulus and the calculated loss modulus at the different mechanical wave frequencies that are used.

The dispersion coefficient, corresponding to the variation of storage and loss modulus with frequency, is also calculated.

The method for predicting also comprises a step S20 of predicting that the subject is at risk of suffering from the obesity related disease based on the determined parameters.

The use of the combination of two techniques applied on the subcutaneous adipose tissue or the epididymal adipose tissue in the method for predicting enables to improve the prediction while keeping the non-invasive property of each technique.

This assertion is shown by the results obtained in the experimental section and in reference to FIGS. 2 to 39. Indeed, it is the first time that the firs analysis and the second analysis is carried out in a murine model of metabolic syndrome. Regarding the first analysis, our results were in agreement with histology. Indeed, the higher levels of non-fat components observed in foz/foz mice epididymal adipose tissue in comparison to WT were also detected on the PDFF and T2* maps with a significant decrease of both PDFF and T2*.

In the liver, changes in fatty acid profile were in agreement with histology and with results of previous fundamental studies. Indeed we observed a significant drop of saturated fatty acid in foz/foz P− mouse in which steatohepatitis lesions are predominant (NAS 7) compared to foz/foz P+in which steatohepatisis moderate (NAS 3-4).

The Applicant's results confirm that adipose tissue fibrosis is more prominent in the intraperitoneal compartment than in the subcutaneous compartments. These compartments have different embryonic origins. Obesity-induced extracellular matrix remodeling can be monitored by the resulting changes in the mechanical properties of the adipose tissue, at least in the epididymal compartment. The lowered dispersion values tend to confirm an implication of inflammatory activity, as we previously observed, in the liver of patients with viral hepatitis, decreased values in response to inflammation. Highly attenuating, strongly dispersive material tends to have elevated values of the dispersion coefficient. Degradation of the material structure, and introduction of mechanical features of different scales from those present in healthy adipose tissue, may thus alter and degrade the ability of tissue to disperse the mechanical waves, and thereby make the tissue behave more like a purely elastic tissue, whereby the frequency has no effect on the mechanical properties. This is in essence what we observed here, where the diseased tissue had flatter frequency response than the healthy, dispersive tissue.

The amount of non-adipose material in the epididymal and subcutaneous compartments closely mirrored the mechanical properties, indicating that mechanical properties are sensitive to extracellular matrix content in the adipose tissue.

It should also be noted that such method enables to obtain a better characterization than a method based on obtaining the stiffness value, because the use of the storage modulus and of the loss modulus enable to better discriminate between obesity related diseases.

This difference notably results from the fact that by only using stiffness values, it is implicitly assumed that each tissue behaves as a solid. This hypothesis neglects the influence of water which is implied in obesity related diseases.

The method for post-processing with both analysis may be used advantageously in other methods, the adaptation to these methods being immediate.

Notably, the method for post-processing may also be adapted for a method for diagnosing an obesity related disease, a method for identifying a therapeutic target for preventing and/or treating an obesity related disease, a method for identifying a biomarker, the biomarker being a diagnostic biomarker of an obesity related disease, a susceptibility biomarker of an obesity related disease, a prognostic biomarker of an obesity related disease or a predictive biomarker in response to the treatment of an obesity related disease and a method for screening a compound useful as a probiotic, a prebiotic or a medicine, the compound having an effect on a known therapeutical target, for preventing and/or treating an obesity related disease.

The embodiments and alternative embodiments considered here-above can be combined to generate further embodiments of the invention.

### EXPERIMENTAL SECTION

Experiments were carried out by an Applicant and are illustrated by the FIGS. 2 to 39. These experiments are detailed below in two subsections relative to the used materials and methods and to the obtained results.

Abbreviations

The following abbreviations are used in the reminder of this section:
MRE: magnetic resonance imaging
AT: adipose tissue
ECM: extracellular matrix
MRI: magnetic resonance imaging
MMP: matrix metalloproteinase
USPIO: ultrasmall paramagnetic iron oxide
HFD: high fat diet
Pio: pioglitazone
G′: storage modulus
G″: loss modulus

γ: wave dispersion coefficient.

Aim of this Experimental Study

Obesity is becoming an important health concern owing to an increased prevalence. Obesity is characterized by a general state of metabolic stress, which is believed to induce chronic inflammation.

AT ECM is extensively remodeled in obesity to accommodate the hypertrophic and hyperplasic response of adipocytes. The remodeling takes place within an inflammatory signaling activation, which can evolve to ECM enrichment, mainly in terms of activated macrophage-secreted collagen VI. This is conserved in small animal and in humans. AT ECM remodeling is also linked to diabetes, as it is involved in the insulin resistance of adipocytes in obesity. Obesity-induced AT ECM remodeling occurs differently in the subcutaneous and in the intraperitoneal compartment, and is often described as more substantial in the latter, where it involves vascular remodeling, as evidenced by altered MMP and angiogenesis factors gene expression and other pro-inflammatory ECM components such as tenascin C and Toll-like receptor 4. In particular, the elastin network is notably different between the intraperitoneal and subcutaneous compartments, with the former being characterized by a dense mesh-like network, while the latter involves more linear depots and associations to macrophages. This consequential interplay between AT ECM and obesity implies that the mechanical properties of AT may be substantially altered in obesity and in response to diabetes-mediated inflammation.

MRI enables to accurately measure quantitative biomarkers of disease. Adipose inflammation can be measured with USPIO, but requires contrast agent injection and relies on a decrease in SNR to detect disease. MRE can be used to measure the mechanical properties of adipose tissue in the breast or in the heel fat pads, but the effect of inflammation on the mechanical properties of adipose tissue has not to date been assessed by MR elastography. Ultrasound elastography was used on subcutaneous adipose tissue, where shear wave velocity was found to increase in presence of adipose tissue fibrosis, and in response to diabetes status. Finally, ex vivo mechanical analyses of adipose tissue samples from obese individuals have revealed significant differences in pseudo-static mechanical properties between subcutaneous and intraperitoneal adipose tissue.

Chemical displacement methods can be used to assess lipid status in vivo, either based on spectroscopy techniques or on multiple echo images. Obesity and diabetes extensively modify the lipid content of AT.

Foz is a mouse model of obesity in which a truncation mutation of the alms1 gene induces spermatogenesis defects, pancreatic β cell islets hyperplasia, and, due to a loss of hypothalamic neuronal ciliae, causes hyperphagia. Upon high-fat diet (HFD) feeding, hyperinsulinemia, hypercholesterolemia, and hyperglycemia quickly take place. HFD-fed foz/foz mice also develop fatty and inflammatory liver (steatohepatitis), and the associated liver necro-inflammatory activity is persistent even after dietary changes. High fat diet-fed foz/foz mice represent an accepted and widely used model of diet-induced obesity and metabolic inflammation and nonalcoholic steatohepatitis.

In the present study the Applicant sought to evaluate, using the magnetic resonance imaging techniques of chemical displacement imaging and MR elastography, the relationship between metabolic stress (and in particular, lipid metabolism), and ECM remodeling via AT biomechanical properties, in the adipose and liver tissue of obese foz/foz mice. The relationship between these markers and diabetes was also investigated through the use of the anti-diabetes drug pioglitazone.

Material and Methods

Experimental Design

Animals were housed under HFD, until the time of MR imaging. MR imaging was carried out on a 7T Bruker Pharmascan equipped with Avancelll spectrometer hardware, a shielded gradient set (300 mT·m⁻¹ maximum gradient amplitude, 110 μs rise time and 80 mm inner diameter) and a ¹H transmit-receive quadrature coil with 40 mm inner diameter for lipid experiments, and an actively decoupled set of ¹H transmit quadrature coil with 50 mm inner diameter and a 8 mm diameter circular surface coil for MRE experiments. MRE and lipid experiments were carried out sequentially. Experiments were carried out by PG (small animal experimentation licence n°A-75-2108) in full compliance with ethical guidelines and with the agreement of the local ethical committee (authorization n°02252.01). Immediately after MRI examination, animals were sacrificed, and their tissue excised and collected in formalin buffer for histopathological examination.

Animals

Foz/foz mice (on a NOD.B10 background) were fed a HFD ad libitum (group denoted “foz/foz P−”), or a HFD supplemented with the anti-diabetic drug, pioglitazone (HFD: Research Diets, USA; pioglitazone 20 mg/kg/day) (group denoted “foz/foz P+”. For MR imaging, animals were anesthetized with isoflurane (0.5-3% in a 50% vol mixture respiration rate and temperature was monitored using a pressure transducer with digital monitoring (model 1025, SA instruments, Stony Brook, United States).

MRI—Fat-Water Separation and Fatty Acid Composition Quantification

The acquisition is first described.

A 2D interleaved multiple echoes spoiled gradient echo sequence with bipolar readout gradients was used. Sixteen echoes were acquired (first echo: 1.59 ms and echo spacing: 0.74 ms). The acquisition parameters were: spoiler gradient duration: 1.5 and 0.8 ms according to readout and phase respectively; TR: 950 ms; Hermitian pulse: 20°, receiver bandwidth: 300 KHz, and 8 signal averages. Geometric parameters were: field of view: (45 mm)², acquisition matrix: 128², 35 transverse slices of 1 mm in thickness. TR and flip angle were adjusted to minimize the T1-related bias and work with suitable signal to noise ratio.

The way the image reconstruction is carried out is now described.

Multiple-echoes complex data were modeled along the echo time with a MR triglyceride model including eight fat spectral components, of which the corresponding number of proton were expressed according to the mean number of double bounds, the mean number of methylene interrupted double bounds per triglyceride and the chain length. Water was considered on resonance and respective chemical shifts were attributed by prior knowledge. The model included a complex field map summarizing both transversal decay and off-resonance effects and a complex error map correcting for the phase errors and the amplitude modulation caused by the bipolar acquisition. Data modelling was performed with an optimization procedure addressing the problem of multiple local minima linked to the chemical ambiguity. The procedure involved a stochastic grid of starting values defining the number of runs and the initialization values for the free parameters in the model. Thereby, no guess values nor prior knowledge of BO field smoothness was required. This step provided water only, and fat only images, as well as parametric maps of proton density fat fraction (PDFF), T2*, saturated, monounsaturated and polyunsaturated fatty acids fractions.

Analysis were performed in the liver, in subcutaneous (SAT), visceral (VAT), and epididymal adipose tissues.

MRI—MR Elastography

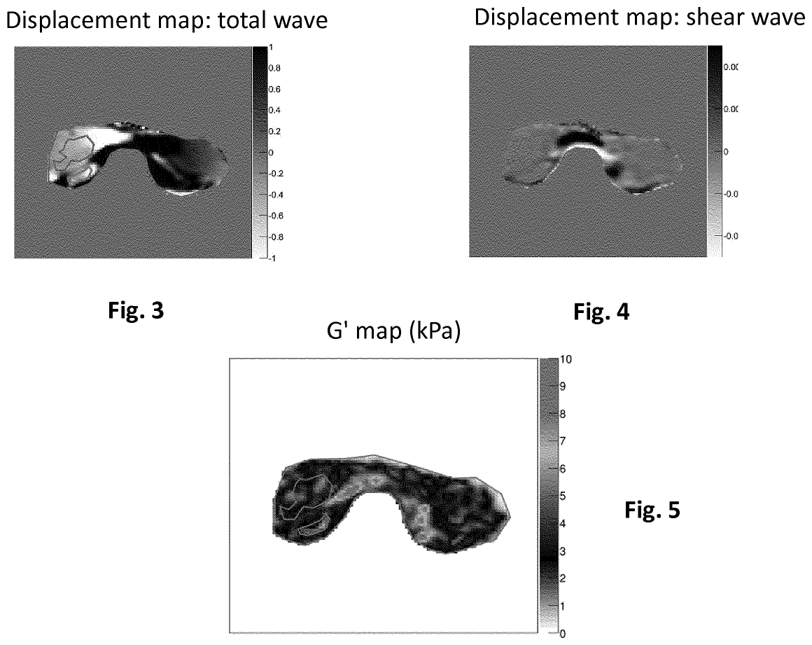

FIGS. 2 to 5 are relative to the MR elastography technique. FIG. 2 illustrates schematically the respiratory motion strategy. A dummy acquisition module was played during animal breathing to enforce synchrony between acquisition and mechanical actuation and magnetic steady state. FIG. 3 illustrates the displacement map including compressional components. This corresponds to the total wave. FIG. 4 shows the divergence-free displacement map. This corresponds to the shear wave. FIG. 5 is an example storage modulus map. Lines illustrate the epididymal region of interest and the subcutaneous region of interest.

MR elastography was performed using a spin echo sequence as previously described. Mechanical actuation was carried out with a homemade piston whose vertical motion was generated by a permanent magnetic vibration exciter (type 4808, Brüel & Kjaar, Naerum, Denmark) located remotely from the MRI, and to which it was rigidly fixated using a carbon fiber rod. Two mechanical frequencies were probed (600 Hz and 1000 Hz) sequentially by recording phase displacement images at four equally spaced time offsets along the mechanical cycle. Repetition time was 1000 ms, and echo time was 29 ms, thus allowing for 6 and 10 periods of motion encoding gradient for the 600 Hz and the 1000 Hz frequencies, respectively. Motion encoding was performed sequentially along the three orthogonal gradient directions with synchronized sinusoidal gradient shapes played at the duty cycle-limited maximal gradient amplitude available on the system (250mT/m and 210 mT/m for the 600 Hz and 1000 Hz mechanical frequencies, respectively).

Respiratory motion compensation was performed by playing a dummy acquisition module during periods of animal breathing in order to maintain synchrony between acquisition and mechanical actuation while maintaining magnetic steady state. Respiration signals originating from the monitoring apparatus 60 were used to identify periods of time in the animal breathing cycles. The cycles are made up of an active part where animal is actively inhaling and exhaling and motion is large, followed by a period of time where the animal is still and motion is small. The system acquisition can only be done on the still part of the cycle so as to prevent respiratory motion from contaminating the images. This was achieved by specifying a delay between detection of a breathing cycle and start of a window of acquisition, and a duration of the window of acquisition. Window of acquisition is defined as a period of time during the breathing cycle where acquisition is enabled. The sequence functions as a continuously running sequence made up of two modules, a dummy module 54 and an acquisition module 56. The dummy module 54 is identical to the acquisition module except for the recording which is turned off, and the image line number incrementation which is also turned off. The dummy module 54 has thus the exact same duration as the acquisition module, thereby enabling to maintain the synchrony between acquisition and mechanical wave. At the end of a module (be it dummy or acquisition), the acquisition system interrogates the monitoring system 58, and conditionally enters either the acquisition module 56 if the acquisition window is open 50, or the dummy module if the acquisition window is closed 52. The duration of the acquisition window was selected to be shorter than the duration of the sequence modules, so as to prevent overlap between the end of an acquisition module and the start of the next breathing motion cycle. This was made possible by having the acquisition module durations shorter than the typical duration of the still parts of the respiration cycles in a normally anesthetized animal.

Nine 300 μm thick contiguous slices were acquired at an in-plane resolution of 300 μm. Phase and magnitude images were recorded. Phase images were unwrapped and used for algebraic inversion of the plane wave propagation equation under assumption of local homogeneity to recover the values of the storage modulus, G′, and of the loss modulus, G″. Compressional components were removed by applying the curl operator to the displacements in order to obtain divergence-free fields. Numerical noise-induced compressional components and other contributions were diminished by gaussian filtering (width 300 μm, span 2 pixels).

Finally, under assumption of isotropy, the directional components of the equations were used for averaging to further reduce the effect of noise.

Regions of interest positioned on the magnitude images were selected on the subcutaneous adipose tissue and in the upper portion of the epididymal fat adipose tissue. Average values for the storage and loss modulus were used for analysis.

Dispersion properties for the storage and loss moduli, γ_{G′} and γ_{G″}, respectively, were approximated by taking the hypothesis of a power law dependence and using the following relationship:

$${γ}_{G} = \frac{ln ( \frac{{G}_{1000}}{{G}_{600}} )}{ln ( \frac{1000}{600} )}$$

where:
G refers to G′ or G″, and
1000 and 600 to the respective frequencies in Hz.

Histologic Analysis

Tissue samples (subcutaneous tissue taken at the level of the lower abdomen, epididymal AT, and liver) were fixated in formalin for 24 hrs at room temperature, then paraffin embedded. Tissue sections were then cut and stained with hematoxylin and eosin. ECM volume fraction was calculated by automatic image histogram-based segmentation of the lipid droplets using an in-house software running in imageJ (imageJ, the National Institutes of Health, Bethesda, United States).

Statistical Analyses

Values were averaged and compared using Mann-Whitney tests. Significance was considered for p values below 0.05.

Results

MRI—Fat-Water Separation and Fatty Acid Composition Quantification

Typical fat and water only images and parametric maps obtained with the present methods in the epididymal adipose tissue are given in FIGS. 6 to 12.

FIGS. 6 to 12 illustrate typical images computed with the first analysis. FIGS. 6 to 12 corresponds respectively to images of water only, of fat only, of PDFF, of T₂*, of SFA, of MUFA and PUFA.

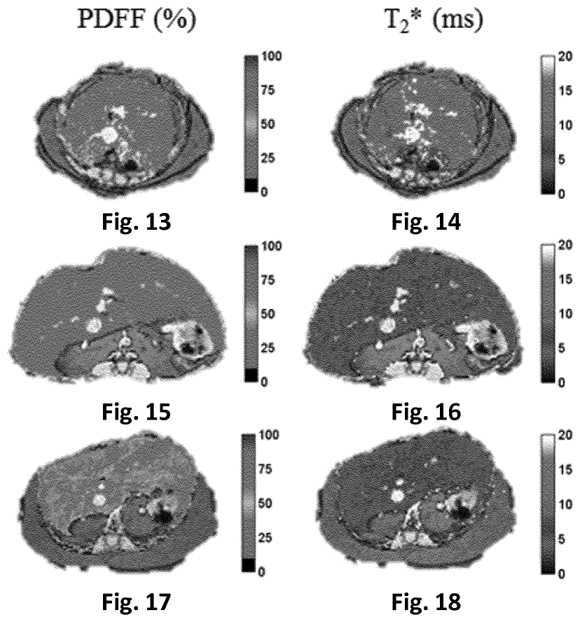

The results for this experiment are illustrated by FIGS. 13 to 24.

FIGS. 13 to 18 are PDFF and T₂* maps of the liver presented in a WT, foz/foz P− and foz/foz P+ mouse. These maps show the difference between the groups. Liver steatosis is more important in foz/foz mice than in WT and among foz/foz mice liver steatosis is more important in mice with pioglitazone. T₂* is also shorter in foz/foz mice than in WT.

In the liver, foz/foz group displayed higher PDFF than in WT (13.0±4.1 vs. 32.9±4.2% p<0.01) and, among the foz/foz group, PDFF was higher in pioglitazone mice (foz/foz P+) than in others (foz/foz P−) (35.3±3.0% vs. 32.9±4.2%; p<0.01). Liver transversal relaxation time was longer in WT (9.3±3.2 ms) than in foz/foz P− (5.0±0.7 ms; p<0.05) and foz/foz P+ (5.7±1.1 ms; p<0.05) groups (FIG. 3). Since fat content was too low in WT livers, FA acid composition was not computed. However, differences in liver fatty acid composition were observed in the foz/foz groups. In foz/foz P− group liver fat was more saturated than in foz/foz P+ (23.8±0.6 vs. 21.8±1.9%; p<0.05). At the opposite, the monounsaturated and polyunsaturated fatty acid fractions were higher in the foz/foz P+ group (53.9±2.4 vs. 52.4±0.3% and 24.4±2.0 23.8±0.8).

Between the groups, adipose tissues displayed transversal relaxation time and PDFF differences. The epididymal adipose tissue displayed a smaller PDFF in the foz/foz P− group (88.4±5.0%) than in WT (96.6±1.2%; p<0.01) and foz/foz P+ (94.4±2.4%; p<0.05) groups and a shorter T2* (24.6±5.6 ms vs.38.0±7.6 ms; p<0.01 and 27.9±6.4 ms in the WT and foz/foz P+ groups respectively) (FIG. 4). In VAT, PDFF was similar between the groups. On the other hand T2* was longer in the WT (26.0±4.1 ms) group than in foz/foz P− (23.0±1.0 ms) and foz/foz P+ (21.2±3.5 ms; p<0.05) groups. No differences were observed in SAT.

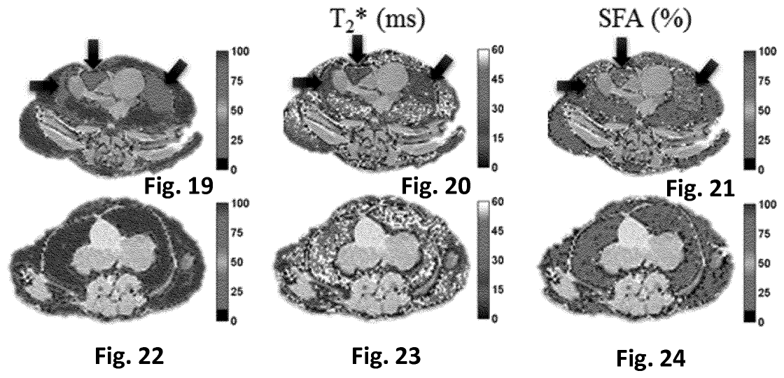

FIGS. 19 to 24 are parametric maps (Proton Density Fat Fraction (PDFF), T₂* and Saturated Fatty Acid fraction (SFA) overlaid on water only images of the epididymal tissue presented in a foz/foz P− and a WT mouse. These maps well illustrated the modification of adipose tissue environment in foz/foz P− mice with a decreased of both PDFF and T₂* suggesting the presence of a water component and the modification of the fatty acid profile with an increase of saturation.

Fatty acid composition differences were also found. SAT was more saturated (p<0.05) in the foz/foz P+ group (27.1±3.4%) than in foz/foz P− (22.6±2.9%) and WT (23.6±1.7%) groups. VAT were more saturated (p<0.05) in the WT (17.8±1.8%) than in the foz/foz P− (15.5±3.3%) and foz/foz P+ (14.2±2.0%) groups. Epididymal adipose tissue were less saturated in WT (25.2±3.2%) than in foz/foz P− (28.8±2.9%) and similar than in the foz/foz P+ group (26.5±3.7%).

Results are summarized in table 1 which is reproduced hereafter.

TABLE 1

| | | Liver | VAT | SAT | Epididymal |
|---|---|---|---|---|---|
| PDFF | WT | 13.0 ± 4.1 | 93.4 ± 1.4 | 91.6 ± 1.4 | 96.6 ± 1.2 |
| | foz/foz P− | 29.7 ± 3.7 | 92.0 ± 0.2 | 91.5 ± 1.2 | 87.8 ± 6.9 |
| | foz/foz P+ | 35.3 ± 3.0 | 92.9 ± 1.4 | 91.4 ± 0.7 | 94.4 ± 2.4 |
| T2* (ms) | WT | 9.3 ± 3.2 | 26.0 ± 4.1 | 24.7 ± 3.8 | 38.0 ± 7.6 |
| | foz/foz P− | 5.0 ± 0.7 | 23.0 ± 1.0 | 23.7 ± 1.0 | 26.2 ± 6.9 |
| | foz/foz P+ | 5.7 ± 1.1 | 21.2 ± 3.5 | 24.7 ± 1.7 | 27.9 ± 6.4 |
| SFA (%) | WT | — | 17.8 ± 1.8 | 23.6 ± 1.7 | 24.1 ± 1.9 |
| | foz/foz P− | 23.8 ± 0.6 | 15.5 ± 3.3 | 22.6 ± 1.9 | 28.8 ± 2.9 |
| | foz/foz P+ | 21.8 ± 1.9 | 14.2 ± 2.0 | 27.1 ± 3.4 | 26.5 ± 3.7 |
| MUFA (%) | WT | — | 57.3 ± 3.7 | 42.6 ± 4.6 | 40.9 ± 2.2 |
| | foz/foz P− | 52.4 ± 0.3 | 61.5 ± 3.4 | 44.6 ± 8.6 | 36.5 ± 9.3 |
| | foz/foz P+ | 53.9 ± 2.4 | 62.6 ± 3.6 | 38.1 ± 7.5 | 39.1 ± 6.3 |
| PUFA (%) | WT | — | 25.0 ± 3.1 | 33.9 ± 3.3 | 35.0 ± 1.1 |
| | foz/foz P− | 23.8 ± 0.8 | 22.9 ± 3.1 | 32.9 ± 5.9 | 34.7 ± 6.8 |
| | foz/foz P+ | 24.4 ± 2.0 | 23.1 ± 2.8 | 34.8 ± 4.2 | 34.5 ± 2.8 |
| G′ at | WT | — | — | 1.41 ± 0.40 | 0.92 ± 0.15 |
| 600 Hz (kPa) | foz/foz P− | — | — | 1.72 ± 0.52 | 1.89 ± 0.82 |
| | foz/foz P+ | — | — | 1.54 ± 0.36 | 1.32 ± 0.25 |
| G″ at | WT | — | — | 0.88 ± 0.16 | 0.76 ± 0.16 |
| 600 Hz (kPa) | foz/foz P− | — | — | 1.02 ± 0.27 | 1.24 ± 0.35 |
| | foz/foz P+ | — | — | 0.99 ± 0.08 | 0.76 ± 0.12 |
| G′ at | WT | — | — | 2.77 ± 0.52 | 2.22 ± 0.31 |
| 1000 Hz (kPa) | foz/foz P− | — | — | 3.28 ± 0.69 | 3.41 ± 0.15 |
| | foz/foz P+ | — | — | 2.69 ± 0.53 | 2.67 ± 0.71 |
| G″ at | WT | — | — | 2.10 ± 0.34 | 1.99 ± 0.50 |
| 1000 Hz (kPa) | foz/foz P− | — | — | 2.03 ± 0.28 | 2.44 ± 0.69 |
| | foz/foz P+ | — | — | 1.97 ± 0.32 | 1.95 ± 0.35 |
| Multifrequency | WT | — | — | 1.37 ± 0.62 | 1.72 ± 0.54 |
| response of G′ | foz/foz P− | — | — | 1.30 ± 0.31 | 1.28 ± 0.91 |
| | foz/foz P+ | — | — | 1.10 ± 0.17 | 1.54 ± 0.65 |
| Multifrequency | WT | — | — | 1.71 ± 0.65 | 1.86 ± 0.20 |
| response of G″ | foz/foz P− | — | — | 1.39 ± 0.35 | 1.34 ± 0.03 |
| | foz/foz P+ | — | — | 1.33 ± 0.19 | 1.59 ± 0.25 |

MR Elastography

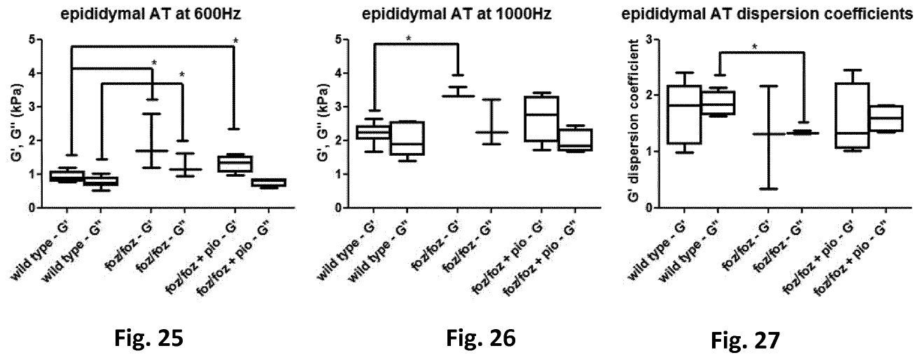

FIGS. 25 to 27 illustrate the mechanical properties in the epididymal adipose tissue compartment. FIG. 25 corresponds to the mechanical properties at 600 Hz, FIG. 26 corresponds to the mechanical properties at 1000 Hz and FIG. 27 illustrates the dispersion properties. Epididymal adipose is found to have higher mechanical properties at both frequencies and lower dispersion properties than wild type. This effect is minimized in foz/foz P+ animals.

In the epididymal adipose tissue, the storage modulus was found to be significantly higher in untreated foz animals than in wild type controls at both tested frequencies (600 Hz: 1.89±0.82 kPa vs. 0.92±0.15 kPa, p<0.05 ; 1000 Hz: 3.41±0.15 kPa vs. 2.22±0.31 kPa, p<0.05, for foz vs. wt, respectively). The loss modulus was also significantly higher in untreated foz animals, but only at the 600 Hz frequency (1.24±0.35 kPa vs. 0.76±0.16 kPa, p<0.05). Animals under pioglitazone treatment had mechanical properties more closely resembling those of controls except for the storage modulus at 600 Hz which remained significantly elevated at 1.32±0.13 kPa (p<0.05). Epididymal adipose tissue had lower average storage modulus dispersion than wild type animals, although the difference was not significant (1.72±0.54 for wild types vs. 1.27±0.91 for the foz animals). Pioglitazone treatment conferred dispersion values closer to those of control animals (1.53±0.65), although these were not significantly different from the values in the wild type or the foz/foz group. Loss modulus dispersion was significantly lower in foz/foz animals than in controls (1.34±0.03 vs. 1.86±0.20, respectively, p<0.05), and this difference was not present in pioglitazone-treated group, which had values not significantly different from those of the control group (1.59±0.24).

In the subcutaneous adipose compartment, no differences were obtained between any groups at any frequency. However, the same tendencies were observed, whereby foz/foz animals had higher single frequency values and lower dispersion values than wild type controls, and pioglitazone-treated foz/foz animals had values intermediate between the control and untreated foz/foz groups (see Table 1).

Histopathological Assessment

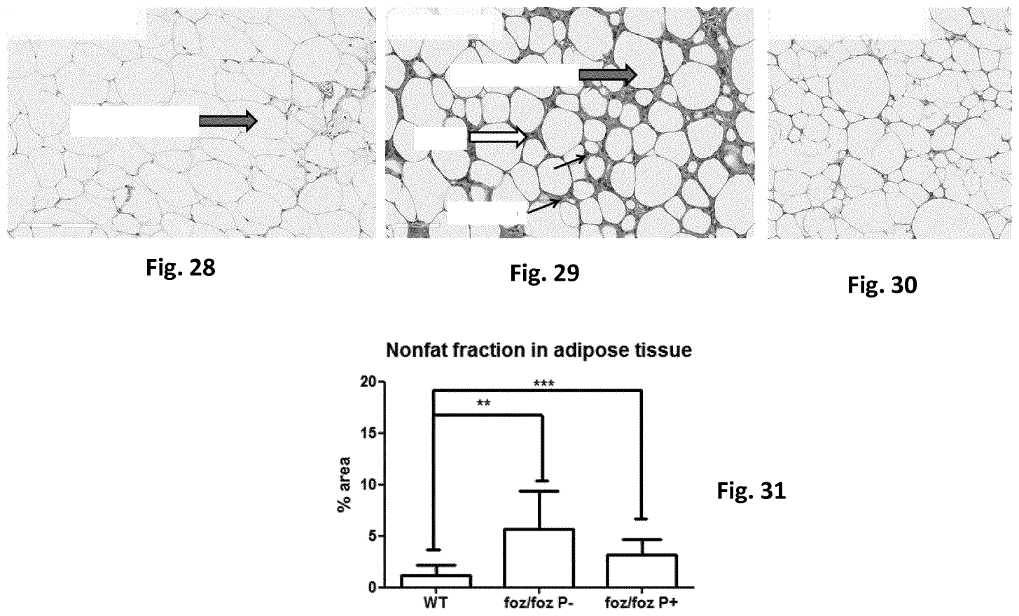

FIGS. 28 to 31 are histopathological analysis of epididymal adipose tissue: hematoxylin and saffron staining (bar: 100 μm) for WT (FIG. 28), foz/foz P− (FIG. 29) and foz/foz P+ (FIG. 30). FIG. 31 is a bar graph which corresponds to the surface occupied by non-fat components (expressed as percentage of field of view surface). Foz/foz animals have significantly higher non-fat component than WT animals. Pioglitazone treatment decreases the non fat fraction, but not to wild type levels.

In epididymal tissue, foz/foz animals displayed significantly higher levels of non-fat components than wild type animals (5.65±3.69% vs. 1.13±1.00%, p<0.01), while pioglitazone treatment resulted in less elevated values (3.15±1.53%), although still significantly higher than wild type controls. The foz/foz tissue appeared to contain a lot of inflammatory signs such as macrophage infiltration, and adipocytes were large in size, whereas in foz/foz under pioglitazone treatment, tissue tended to be less inflammatory, and adipocyte sizes were more diverse.

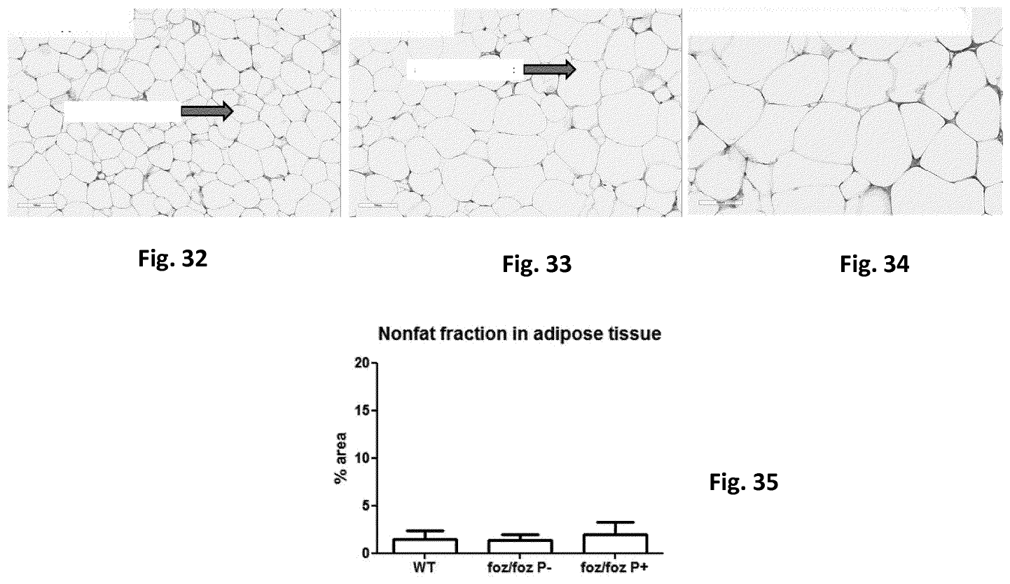

FIGS. 32 to 35 are histopathological analysis of subcutaneous adipose tissue: hematoxylin and saffron staining (bar: 100 μm) for WT (FIG. 32), foz/foz P− (FIG. 33) and foz/foz P+ (FIG. 34). FIG. 35 is a bar graph which corresponds to the surface occupied by non-fat components (expressed as percentage of field of view surface). WT, foz/foz P− and foz/foz P+ all have similar levels of non-fat adipose tissue.

In subcutaneous tissue, no significant differences were found in non-fat fraction of adipose tissue (wild type: 1.48±0.93, foz/foz: 1.40±0.61, foz/foz+pio: 2.01±1.21). In foz/foz mice, adipocytes tended to be large in size, and crown-like structures of macrophages encircling adipocytes were visible. foz/foz animals under pioglitazone treatment had a large variance in adipocyte size, and had enhanced inflammatory intercellular content.

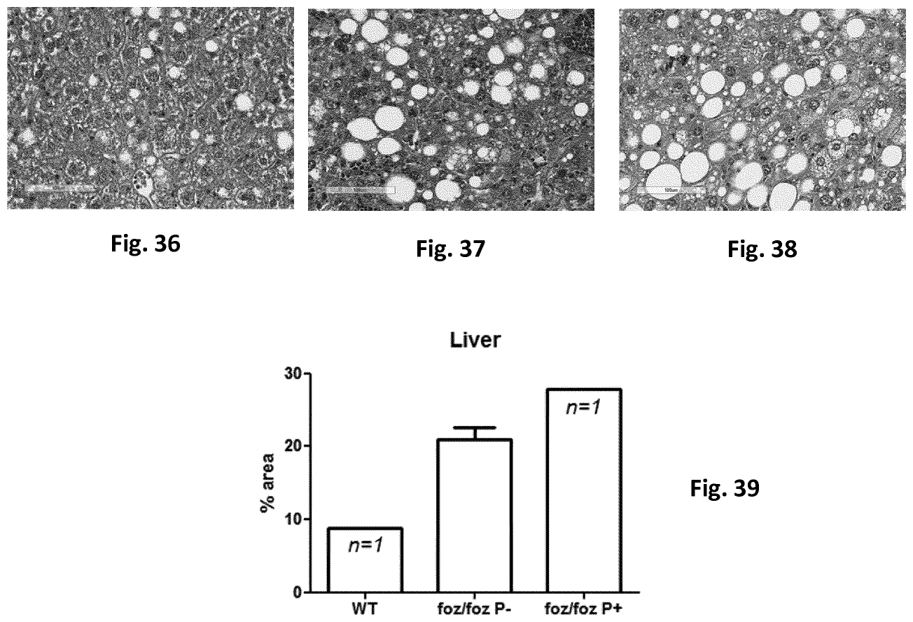

FIGS. 36 to 39 are liver tissue sections colored with hematoxylin and saffron (bar: 100 μm) for WT (FIG. 36), foz/foz P− (FIG. 37) and foz/foz P+ (FIG. 38). FIG. 39 is a bar graph which corresponds to the surface occupied by non-fat components (expressed as percentage of field of view surface) for WT, foz/foz P− and foz/foz P+ animals. Foz/foz animals tended to show more signs of inflammatory-mediated injury, while pioglitazone treatment diminished these effects.

In the liver, wild type animals displayed limited steatosis and no evidence of steatohepatitis. foz/foz animals were positive for steatohepatitis, with hepatocellular injury, apoptosis, inflammation and ballooning readily visible and equivalent to a NAS score of 7. Foz/foz animals under pioglitazone treatment displayed localized steatosis with both a macro- and a micro-vesicular component, little to no cellular death and little inflammation signs, and were equivalent to a NAS score of 3-4.

1. A method for predicting that a subject is at risk of suffering from an obesity related disease, the method for predicting at least comprising the step of:
carrying out the steps of a method for post-processing images of the subject, to obtain determined parameters,
the method for post-processing being a method for post-processing two series of images of tissues of a subject, the tissues being either the subcutaneous adipose tissue or the epididymal adipose tissue, the first series of images being taken by a magnetic resonance imaging technique, the magnetic resonance imaging technique involving successive echoes of a multiple-gradient echo sequence with bipolar readout gradients and the second series of images being taken by using a multifrequency magnetic resonance elastography technique, the method for post-processing comprising the substeps of:
quantifying the proportion of unsaturated fatty acids and the proportion of saturated fatty acids in the tissues of the subject based on a first analysis applied on the first series of images and
calculating the values of the storage modulus and the loss modulus in the tissues of the subject based on a second analysis applied on the second series of images,
the determined parameters being the quantified proportion of unsaturated fatty acids, the quantified proportion of saturated fatty acids, the calculated values of the storage modulus and the calculated loss modulus,
predicting that the subject is at risk of suffering from the obesity related disease based on the determined parameters.

2. A method for treating an obesity related disease, the method for diagnosing at least comprising the step of:
carrying out the steps of a method for post-processing images of the subject, to obtain determined parameters,
the method for post-processing being a method for post-processing two series of images of tissues of a subject, the tissues being either the subcutaneous adipose tissue or the epididymal adipose tissue, the first series of images being taken by a magnetic resonance imaging technique, the magnetic resonance imaging technique involving successive echoes of a multiple-gradient echo sequence with bipolar readout gradients and the second series of images being taken by using a multifrequency magnetic resonance elastography technique, the method for post-processing comprising the substeps of:
quantifying the proportion of unsaturated fatty acids and the proportion of saturated fatty acids in the tissues of the subject based on a first analysis applied on the first series of images and
calculating the values of the storage modulus and the loss modulus in the tissues of the subject based on a second analysis applied on the second series of images,
the determined parameters being the quantified proportion of unsaturated fatty acids, the quantified proportion of saturated fatty acids, the calculated values of the storage modulus and the calculated loss modulus, and
administering a medicine for the obesity related disease determined based on the determined parameters.

3. A method for identifying a therapeutic target for preventing and/or treating an obesity related disease, the method comprising the steps of:
carrying out the steps of a method for post-processing images of a first subject, to obtain first determined parameters,
the method for post-processing being a method for post-processing two series of images of tissues of a subject, the tissues being either the subcutaneous adipose tissue or the epididymal adipose tissue, the first series of images being taken by a magnetic resonance imaging technique, the magnetic resonance imaging technique involving successive echoes of a multiple-gradient echo sequence with bipolar readout gradients and the second series of images being taken by using a multifrequency magnetic resonance elastography technique, the method for post-processing comprising the substeps of:
quantifying the proportion of unsaturated fatty acids and the proportion of saturated fatty acids in the tissues of the subject based on a first analysis applied on the first series of images and
calculating the values of the storage modulus and the loss modulus in the tissues of the subject based on a second analysis applied on the second series of images,
the first determined parameters being the quantified proportion of unsaturated fatty acids, the quantified proportion of saturated fatty acids, the calculated values of the storage modulus and the calculated loss modulus, the first subject being a subject suffering from the obesity related disease,
carrying out the steps of the method for post-processing images of a second subject, to obtain second determined parameters,
the second determined parameters being the quantified proportion of unsaturated fatty acids, the quantified proportion of saturated fatty acids, the calculated values of the storage modulus and the calculated loss modulus, the second subject being a subject not suffering from the obesity related disease,
selecting a therapeutic target based on the comparison of the first and second determined parameters.

4. A method for identifying a biomarker, the biomarker being a diagnostic biomarker of an obesity related disease, a susceptibility biomarker of an obesity related disease, a prognostic biomarker of an obesity related disease or a predictive biomarker in response to the treatment of an obesity related disease, the method comprising the steps of:
carrying out the steps of a method for post-processing images of a first subject, to obtain first determined parameters,
the method for post-processing being a method for post-processing two series of images of tissues of a subject, the tissues being either the subcutaneous adipose tissue or the epididymal adipose tissue, the first series of images being taken by a magnetic resonance imaging technique, the magnetic resonance imaging technique involving successive echoes of a multiple-gradient echo sequence with bipolar readout gradients and the second series of images being taken by using a multifrequency magnetic resonance elastography technique, the method for post-processing comprising the substeps of:
quantifying the proportion of unsaturated fatty acids and the proportion of saturated fatty acids in the tissues of the subject based on a first analysis applied on the first series of images and
calculating the values of the storage modulus and the loss modulus in the tissues of the subject based on a second analysis applied on the second series of images,
the first determined parameters being the quantified proportion of unsaturated fatty acids, the quantified proportion of saturated fatty acids, the calculated values of the storage modulus and the calculated loss modulus, the first subject being a subject suffering from the obesity related disease,
carrying out the steps of the method for post-processing images of a second subject, to obtain second determined parameters,
the second determined parameters being the quantified proportion of unsaturated fatty acids, the quantified proportion of saturated fatty acids, the calculated values of the storage modulus and the calculated loss modulus, the second subject being a subject not suffering from the obesity related disease,
selecting a biomarker based on the comparison of the first and second determined parameters.

5. A method for screening a compound useful as a probiotic, a prebiotic or a medicine, the compound having an effect on a known therapeutical target, for preventing and/or treating an obesity related disease, the method comprising the steps of:
carrying out the steps of a method for post-processing images of a first subject, to obtain first determined parameters,
the method for post-processing being a method for post-processing two series of images of tissues of a subject, the tissues being either the subcutaneous adipose tissue or the epididymal adipose tissue, the first series of images being taken by a magnetic resonance imaging technique, the magnetic resonance imaging technique involving successive echoes of a multiple-gradient echo sequence with bipolar readout gradients and the second series of images being taken by using a multifrequency magnetic resonance elastography technique, the method for post-processing comprising the substeps of:
quantifying the proportion of unsaturated fatty acids and the proportion of saturated fatty acids in the tissues of the subject based on a first analysis applied on the first series of images and
calculating the values of the storage modulus and the loss modulus in the tissues of the subject based on a second analysis applied on the second series of images,
the first determined parameters being the quantified proportion of unsaturated fatty acids, the quantified proportion of saturated fatty acids, the calculated values of the storage modulus and the calculated loss modulus, the first subject being a subject suffering from the obesity related disease and having received the compound,
carrying out the steps of the method for post-processing images of a second subject, to obtain second determined parameters,
the second determined parameters being the quantified proportion of unsaturated fatty acids, the quantified proportion of saturated fatty acids, the calculated values of the storage modulus and the calculated loss modulus, the second subject being a subject suffering from the obesity related disease and not having received the compound,
selecting a compound based on the comparison of the first and second determined parameters.

6. The method according to claim 1, wherein each image of the first series of images associates to each pixel of the image the amplitude of the measured signal in the magnetic resonance imaging technique and the phase of the measured signal in the magnetic resonance imaging technique, the first analysis applied on the first series of images at the substep of quantifying comprising:
unwrapping the phase of each image, to obtain unwrapped images,
extracting a complex signal over echo time for at least one pixel of the unwrapped images, to obtain at least one extracted complex signal, and
calculating fat characterization parameters by using a fitting technique applied on a model,
the model being a function which associates to a plurality of parameters each extracted complex signal, the plurality of parameters comprising at least two fat characterization parameters, the magnitude error generated by the use of the bipolar readout gradients and the phase error generated by the use of the bipolar readout gradients,
the fitting technique being a non-linear least-square fitting technique using pseudo-random initial conditions, and
quantifying the proportion of unsaturated fatty acids and proportion of saturated fatty acids in the area of the subject based on the calculated fat characterization parameters.

7. The method according to claim 6, wherein the fat characterization parameters are chosen in the group consisting of the number of double bounds, the number of methylene-interrupted double bounds and the chain length.

8. The method according to claim 6, wherein the first analysis applied on the first series of images at the substep of quantifying further comprises the step of:
determining the phase jump in the phase between two images, the first image being taken at a first echo and the second image being taken at a second consecutive echo,
comparing the phase jump with a threshold value, and
correcting the phase value when the phase jump is superior or equal to the threshold value.

9. The method according to claim 6, wherein the model further depends on the complex intensity of water, the complex intensity of fat and a complex field map taking into account the effect of transversal relaxivity rate and the field inhomogeneity in the magnetic field used in the magnetic resonance imaging technique.

10. The method according to claim 6, wherein the first analysis applied on the first series of images at the substep of quantifying further comprises determining the proton density fat fraction and the fatty acid composition based on the calculated fat characterization parameters.

11. The method according to claim 6, wherein each image of the second series of images associates to each pixel of the image the amplitude of the measured signal in the magnetic resonance elastography technique and the phase of the measured signal in the magnetic resonance elastography technique, the second analysis at the substep of calculating comprises:
unwrapping the phase of each image, to obtain unwrapped images,
obtaining the propagation equation of a wave in the imaged tissues based on the unwrapped images, and
recovering the values of the storage modulus and the loss modulus in the tissues of the subject by using the propagation equation and the amplitude of the measured signal.

12. The method according to claim 6, wherein the substep of calculating the values of the storage modulus and the loss modulus in the tissues of the subject based on a second analysis applied on the second series of images further comprises determining the dispersion coefficients of the calculated values of the storage modulus, the calculated loss modulus and the determined parameters further comprises the dispersion coefficients of the calculated values of the storage modulus, the calculated loss modulus.

13. the method according to claim 12, wherein the dispersion coefficients are respectively the variation of the values of the storage modulus with frequency and the variation of the values of the loss modulus with frequency.

14. A computer program product comprising a computer readable medium, having thereon a computer program comprising program instructions, the computer program being loadable into a data-processing unit and adapted to cause execution of a method according to any one of the claims 1 to 5 when the computer program is run by the data processing unit.

15. A computer readable medium having encoded thereon a computer program according to claim 14.
